(19) United States
(12) Patent Application Publication
(10) Pub. No.: US 2024/0052296 A1
(43) Pub. Date: Feb. 15, 2024

(54) METHOD FOR PRODUCING COLANIC ACID USING RECOMBINANT ESCHERICHIA COLI

(71) Applicant: Korea University Research and Business Foundation, Seoul (KR)

(72) Inventors: Kyoung Heon KIM, Seoul (KR); Eun Ju YUN, Seoul (KR); Yoon Ho CHO, Seoul (KR); So Ra YU, Seoul (KR)

(73) Assignee: Korea University Research and Business Foundation, Seoul (KR)

(21) Appl. No.: 17/819,767

(22) Filed: Aug. 15, 2022

(51) Int. Cl. C12N 1/20; C12N 15/10

(52) U.S. Cl. CPC C12N 1/20; C12N 15/102; C12N 2500/34; C12N 2510/02

(57) ABSTRACT

The present invention relates to an optimum method for mass-producing colanic acid by changes in the genetic factors of the strain and the environmental factors of the strain culture. The biological production amount of colanic acid can be significantly increased using the recombinant strain and culture conditions of the present invention.

### INCORPORATION BY REFERENCE OF SEQUENCE LISTING

The content of the electronically submitted sequence listing, file name: Q278953sequencelistingasfiled.XML; size: 6,001 bytes; and date of creation: Aug. 15, 2022, filed herewith, is incorporated herein by reference in their entirety.

### BACKGROUND

### 1. Field of the Invention

The present invention relates to a method for mass-producing colanic acid using a recombinant E. coli.

### 2. Discussion of Related Art

Colanic acid is one of the extracellular polysaccharides with a negative charge and a molecular weight of 3.4 kDa, and is known to be produced while various bacteria belonging to the family Enterobacteriaceae form a biofilm and are growing. Colanic acid is presumed to play an important role in the formation of the three-dimensional structure of the biofilm, and imparts resistance to phage infection, resistance to environmental factors such as osmotic pressure, dehydration, low temperature, and oxidative stress, resistance to antibiotics, and the like to bacteria present therein. Colanic acid forms a structure in which six sugars are repeatedly present, and the six sugars are two fucoses, two galactoses, one glucose, and one glucuronic acid, respectively. Additionally, it has acetic acid and pyruvic acid as residues. Fucose, which is one of these monomeric sugars, is a rare sugar that cannot be readily obtained, but has various physiologically active functions, and thus is widely used in the fields of food and medicine and cosmetics. As a food material, fucose is used as a coagulant, film-forming agent, gel-forming agent, and emulsion stabilizer due to its high water-binding capacity, and is also used as a diet sugar due to its low calorie content. As a pharmaceutical product, fucose is used as an anti-inflammatory agent, anti-cancer agent, an immune enhancer, and the like, and is also used as a material for cosmetics due to its effects such as a whitening effect, a moisturizing effect, promotion of skin cell regeneration, and anti-aging. However, since the method of obtaining fucose is difficult compared to many such uses and the yield is low, the price is very high. However, since colanic acid, which is targeted for maximum production here, has a fucose as a monomer, and its amount also accounts for about ⅓ of the total mass, mass production of colanic acid could be used to produce fucose. Further, as recent research results have revealed various bioactive functions of colanic acid itself, the utility value of colanic acid is increasing.

However, there are not many studies on strain and medium optimization for mass production of colanic acid using microorganisms. Thus, as a result of removing a waaF gene and culturing the gene in Escherichia coli by optimizing culture medium conditions in a previous study, the present inventors confirmed that the production of colanic acid was improved (Korean Patent Application No. 10-2019-0049226). Through this, it was confirmed that genetic and environmental conditions are very important for improving the concentration and yield of colanic acid produced by E. coli.

### RELATED ART DOCUMENT

### Non-Patent Documents

(Non-Patent Document 1) Front. Microbiol. 6:496(2015), Bacterial exopolysaccharides: biosynthesis pathways and engineering strategies

### SUMMARY OF THE INVENTION

The present inventors have intended to develop a strain optimum for the production of colanic acid and mass-produce colanic acid through changes in various environmental factors including oxidative stress, osmotic stress and pH conditions, in order to improve the production yield of colanic acid from E. coli.

To date, fucose has been usually produced from fucoidan present in brown algae by acid hydrolysis and purification processes. However, not only the content of fucoidan present in brown algae is 3 to 5%, which is very low, but also fucose present in fucoidan is substituted with a sulfuric acid group, so there is a disadvantage in that the production yield is low because a process of additionally removing the sulfuric acid group needs to be performed (Ale MT et al (2013) RSC Adv. 3: 8131-8141). In contrast, when fucose is obtained from colanic acid produced through fermentation of recombinant E. coli, there is an advantage in that the production yield of fucose is relatively high compared to brown algae. Therefore, in the present invention, it was confirmed whether colanic acid was produced from recombinant E. coli by additionally introducing a gene related to the production of colanic acid, which is a genetic factor, into an E. coli JM109 (DE3) strain in which a waaF gene, which is a strain prepared in a previous invention, was deleted, and introducing various oxidative stress, osmotic stress and pH conditions corresponding to environmental factors. As a result, it was confirmed that an rcsF gene and oxidative stress and pH conditions are factors affecting the production of colanic acid.

Thus, the present invention provides a method for producing colanic acid, the method including:
preparing a gene recombinant E. coli JM109 strain by removing a waaF gene from an E. coli JM109 strain and introducing an rcsF gene; and
culturing the prepared gene recombinant E. coli JM109 strain in a fermentation medium.

A publicly-known gene manipulation technique for removing or introducing a specific gene may be used without limitation in order to remove a waaF gene from an E. coli JM109 strain corresponding to a common strain and introduce an rcsF gene. In an exemplary embodiment of the present invention, a recombinant E. coli JM109 strain in which a waaF gene was removed was prepared using a lambda-red recombination technique illustrated in FIG. 1.

rcsF, which was additionally introduced into E. coli, is a gene encoding an outer membrane protein RcsF. RcsF is known to play a role in sensing cell membrane stress and transmitting signals (Majdalani N et al (2005) J Bacteriol. 187(19): 6770-6778). In particular, E. coli cells are known to activate the production of colanic acid through an RcsCDB phosphorelay system (Ren G et al (2016) J Bacteriol. 198(11): 1576-1584).

In a specific embodiment, it was confirmed that when an rcsF gene was additionally introduced into E. coli in which a waaF gene was deleted, the concentration and yield of colanic acid were about 1.4-fold and about 1.2-fold higher, respectively, than those of colanic acid produced by E. coli in which the waaF gene was deleted (FIG. 2).

The present inventors selected variables that have the greatest effect on the production amount of colanic acid among various components contained in a medium mixture and conveniently and efficiently selected the optimum conditions of a strain fermentation medium by minimizing the number of enormous experimental conditions, using a statistical method such as fractional factorial design, a steepest ascent method and a response surface methodology in order to optimize the medium (Korean Patent Application No. 10-2019-0049226).

Specifically, it was confirmed that when glucose was used as a carbon source and tryptone was used as a nitrogen source by confirming the colanic acid production yield of the strain for various carbon sources and nitrogen sources in order to optimize the composition and concentration of the fermentation medium, the colanic acid production yield was excellent. Accordingly, the fermentation medium may additionally further include glucose, tryptone, sodium phosphate (Na₂HPO₄), sodium chloride (NaCl), magnesium sulfate (MgSO₄), calcium chloride (CaCl₂) and potassium phosphate (KH₂PO₄).

The fermentation medium may include 10 to 30 g/l glucose, 7 to 15 g/l sodium phosphate, 1 to 5 g/l potassium phosphate, 0.1 to 1 g/l sodium chloride, 1 to 5 g/l tryptone, 0.1 to 0.5 g/l magnesium sulfate and 0.005 to 0.02 g/l calcium chloride.

In specific embodiments of the present invention, an optimized fermentation medium may include 20 g/l glucose, 10.62 g/l sodium phosphate, 3.00 g/l potassium phosphate, 0.5 g/l sodium chloride, 2.63 g/l tryptone, 0.24 g/l magnesium sulfate and 0.011 g/l calcium chloride.

A recombinant E. coli strain may be cultured in the fermentation medium at 20 to 30° C., and specifically, at 25° C.

It is possible to further include pre-culturing the recombinant E. coli strain in an LB medium before culturing the recombinant E. coli strain in the fermentation medium.

The LB medium includes agar, and the pre-culturing step may be performed at 30 to 40° C.

The culturing step may be performed in an incubator including a liquid culture medium. In the present invention, the incubator may include various flasks and reactors capable of culturing microorganisms, but is not limited thereto. When the strain is cultured in the incubator, a culture volume (working volume) may be 10 to 50% of the total volume of the incubator, preferably 20 to 30%.

As an exemplary embodiment for achieving the above-described objects, the recombinant E. coli JM109 strain in which the waaF gene is removed and the rcsF gene is introduced may be cultured in a culture medium under conditions of pH 7. In a specific embodiment, as a result of culturing the strain under neutral conditions of pH 7, a colanic acid concentration (4351.6 mg/L) and yield (5180.4 mg/g DCW) were obtained.

As an exemplary embodiment for achieving the above-described objects, it is possible to additionally include adding oxidative stress during the culturing of the recombinant E. coli JM109 strain in which the waaF gene is removed and the rcsF gene is introduced.

As used herein, the term “oxidative stress” refers to stress in which a high content of oxidizing material is present in the surrounding environment of cells, and the possibility that components such as lipids contained in the cell membrane can be oxidized is increased due to the oxidizing material. Although the oxidative stress can be relieved by secreting an antioxidant material capable of reducing the oxidative stress in vivo, the antioxidant material should be artificially added under in vitro conditions. In particular, since various metabolisms such as growth and proliferation are inhibited by oxidizing lipid components of the cell membrane of the strain when oxidative stress is added to the culture environment of the strain, the growth of the strain decreases as the level of oxidative stress in the culture environment increases, and the strain dies when the level exceeds a certain level.

In the present invention, the oxidative stress may be formed by adding an oxidizing agent to the culture environment of the strain, and specifically, hydrogen peroxide (H₂O₂), a superoxide ion (Of), a hydroxyl radical (OH), hypochlorous acid (HOCl), and the like may be used alone or in combination, and the addition amount of oxidizing agent is also not particularly limited, but the oxidizing agent may be added to the medium of the strain at a concentration of preferably 0.5 to 100 mM, more preferably 5 to 50 mM, and most preferably 10 to 30 mM.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 schematically illustrates a λ-red mediated recombination method for removing a waaF gene from an E. coli JM109 strain;

FIG. 2 illustrates the results of comparing the concentration and yield of the colanic acid produced by a parental strain and recombinant E. coli. (A) Growth curve, (B) Concentrations and yields of colanic acid obtained from E. coli JM109(DE3), E. coli rcsF, E. coli ΔwaaF, E. coli ΔwaaF+pET21a, and E. coli ΔwaaF+rcsF strains;

FIG. 3 illustrates the effect of sodium chloride on the concentration and yield of colanic acid produced by the E. coli Δ waaF+rcsF strain. (A) Growth curve, (B) Concentration and yield of colanic acid obtained by the E. coli Δ waaF+rcsF strain when the concentration range of sodium chloride was set to 0 to 300 mM and 0, 10, 25, 50, 100, 200, and 300 mM were each added to the medium;

FIG. 4 illustrates the effect of hydrogen peroxide on the colanic acid produced by the E. coli ΔwaaF+rcsF strain. (A) Growth curve, (B) Concentration and yield of colanic acid produced by the E. coli ΔwaaF+rcsF strain when hydrogen peroxide was put into the medium in a range of 1 to 50 mM (*, p<0.05; **, p<0.01);

FIG. 5 illustrates the results of confirming the optimum pH of a medium for allowing E. coli ΔwaaF+rcsF to produce the maximum amount of colanic acid. (A) Growth curve, (B) Concentration and colanic acid produced by the E. coli ΔwaaF+rcsF bacteria that grew when the pH range of the medium was set from 5 to 8;

FIG. 6 illustrates the results of comparing the effects on colanic acid produced by recombinant E. coli when the optimum pH and hydrogen peroxide concentration for the maximum production of colanic acid from E. coli ΔwaaF+rcsF was added to the medium at one time. (A) Growth curve, (B) Concentration and yield of colanic acid produced by the E. coli ΔwaaF+rcsF bacteria when 20 mmM hydrogen peroxide was added and when 20 mmM hydrogen peroxide was not added under the conditions in which the medium was maintained at pH 7;

FIG. 7 illustrates the results of comparing the changes in production concentration and yield of colanic acid according to gene modifications of E. coli and environmental changes; and

FIG. 8 illustrates the results of comparing changes in the production yield of colanic acid according to changes in culture volume when recombinant E. coli was fermented in an incubator.

### DETAILED DESCRIPTION OF EXEMPLARY EMBODIMENTS

Hereinafter, the present invention will be described in detail through the Examples. However, the following Examples are only for exemplifying the present invention, and the content of the present invention is not limited by the following Examples.

### [Example 1] Preparation of Recombinant E. Call

1. Removal of waaF Gene

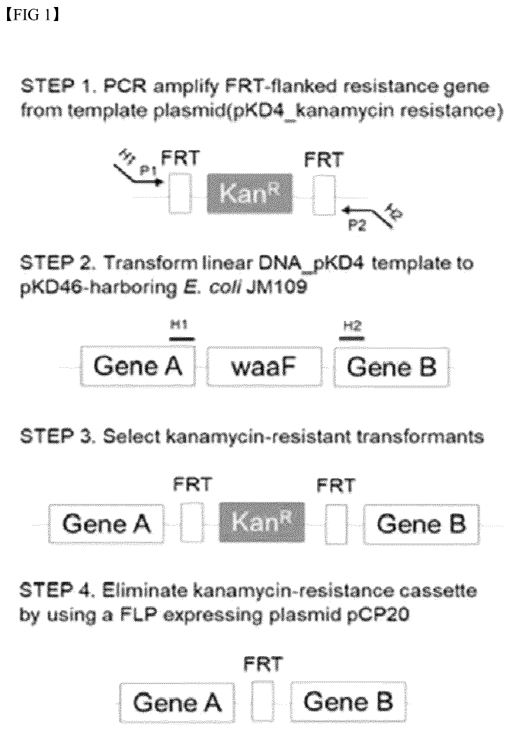

The strain used in the present invention was E. coli JM109 ΔwaaF, and a waaF gene was removed from a general E. coli JM109 strain. The strategy used for gene removal is a λ-red recombination technique and is briefly introduced in FIG. 1, and the detailed process is as follows.

1) A pKD4 plasmid was used to amplify the sequence fragments including a kanamycin resistance gene between flippase recognition targets (FRTs) and the upstream and downstream genes having homology with the flanking regions of waaF by PCR.

The primer sets used to amplify the sequence fragments are as follows:

Forward primer:
(SEQ ID NO: 1)
5′-ATGGTGCCGTCCATTATTATCGCGGATGCCGGAAGTTAACGAAGCTA
TTCTTGTGTAGGCTGGAGCTGCTTC-3′
and
reverse primer:
(SEQ ID NO: 2)
5′-GATAACCCTCCGCAGCGTCACCTTTACGCACTTTGTGATAGCCGGTA
ATCATGGGAATTAGCCATGGTCC-3′.

The underlined sequence sites of the above primers indicate the homologous recombination sites of waaF. 2) A pKD46 plasmid was inserted into E. coli JM109.

3) After a linear DNA template amplified in Process 1) was inserted into E. coli JM109 into which the pKD46 plasmid was inserted, the pKD46 plasmid was expressed.

4) A strain in which only a waaF gene was removed from the existing E. coli JM109 was completed by inserting a pCP20 plasmid expressing a flippase recombinant protein, and then expressing the flippase recombinant protein.

2. Gene Cloning for Introduction of resF Gene

In the present invention, E. coli was additionally manipulated to investigate the synergistic effect of waaF deletion and rcsF expression in order to improve the yield of colanic acid. To introduce an rcsF gene, E. coli K-12 MG1655 (DE3) having an rcsF gene sequence was grown in an LB medium at 37° C. After the E. coli was cultured for 16 hours, cells were centrifuged for 15 minutes by setting a centrifuge at 10,170×g and 4° C. to collect the cells. A commercial DNA extraction kit (GeneAll Biotechnology Co., Seoul) was used to extract the genomic DNA of the bacteria. A desired gene rcsF region was amplified using primers having the following sequences: rcsF-N, 5′-AAAGGATCCATGCGTGCTTTACCGATCTGTTT-3 ‘ (SEQ ID NO: 3); and rcsF-C, 5’-AAACTCGAGTCATTTCGCCGTAATGTTAAGCG-3 (SEQ ID NO: 4). A PCR product and a pET21a plasmid (Novagen, Madison, WI, USA) were digested using restriction enzymes BamHI and Xhol, and then rcsF and pET21a were ligated using a T4DNA ligase (BioLabs, MA, USA). The plasmid was inserted into the bacteria by electric shock. Then, colonies were selectively taken using an LB-ampicillin medium.

TABLE 1

| Strain/Plasmid | Relevant description | Reference |
|---|---|---|
| Strain | | |
| E. coli DH5α | F-, [symbol] lacZΔM15, endA, [symbol] recA [symbol] , [symbol] sdR17(rK [symbol] mK [symbol] ), | Invitrogen |
| | supE44, [symbol] gyrA96, relA [symbol] , Δ(lacZYA-argF)U169 | (Carlsbad, CA, USA) |
| E. coli JM109(DE3) | F [symbol] traD36 proA + B + lac [symbol] Δ(lacZ)M15 [symbol] (lac-proAB), | NEB |
| | [symbol] V44 e14-gyrA96 recA [symbol] relA [symbol] endA [symbol] [symbol] 1 [symbol] R17 | (Ipswich, MA, USA) |
| E. coli [symbol] | JM109(DE3) ΔwaaF (waaF gene was deleted) | This study |
| E. coli pET21a | JM109(DE3) harboring pET21a | This study |
| E. coli rcsF | JM109(DE3) expressing rcsF | This study |
| E. coli ΔwaaF + pET21a | JM109(DE3) ΔwaaF harboring pET21a | This study |
| E. coli ΔwaaF + rcsF | JM109(DE3) ΔwaaF expressing rcsF | This study |
| Plasmid | | |
| pET21a | T7 promoter with MCS, pBR322 replicon, AMP [symbol] | Novagen |
| | | (Burlington, MA, USA) |
| [symbol] | pET21a harboring rcsF | This study |

[symbol]

indicates data missing or illegible when filed

### [Example 2] Measurement of Absorbance at 600 nm for Growth Curve of Recombinant E. coli

In order to measure the absorbance of a viscous culture medium, the medium was diluted with tertiary distilled water and then absorbance was measured. The optical density of cells was measured at 600 nm (OD₆₀₀) at 6 hour intervals during 48 hours of fermentation using a spectrophotometer (SmartSpec Plus Spectrophotometer, Bio-Rad, Hercules, CA, USA).

### [Example 3] Quantification of Colanic Acid, which is an Extracellular Polysaccharide Produced by Recombinant E. coli

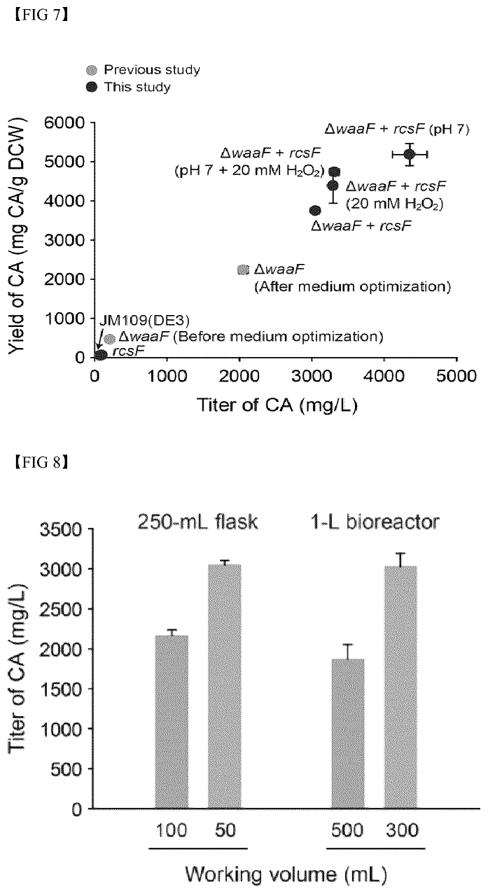

Bacteria were cultured at 25° C. and 200 rpm for 48 hours by varying the culture volume in an incubator (a 250-mL flask or a 1-L fermenter (bioreactor)) (FIG. 8), and cultured by setting the air flow rate at 200 ml/min during culture in the 1-L fermenter (bioreactor). 35 ml of the bacterial culture solution was collected at 48 hours, and then put into a 50-ml conical tube. In order to inactivate the enzyme that degrades extracellular polysaccharides and completely separate colanic acid present on the cell surface, and inactivation was performed at 95° C. for 10 minutes. Then, after cooling on ice, the tube was centrifuged using a centrifuge in order to separate a supernatant from the 35 ml bacterial culture solution. After centrifugation at 10,000×g and 4° C. for 30 minutes, 10 ml of the supernatant was put into 30 ml of cold ethanol to precipitate colanic acid. After sufficient precipitation at 4° C. for about 16 hours, a precipitate was obtained by centrifugation at 4° C. and 10,000×g for 30 minutes using a centrifuge. Then, after ethanol, which is the supernatant, was completely removed, the precipitate was obtained. In order to remove the remaining ethanol, the remaining ethanol was volatilized while the tube was placed on ice. Colanic acid was quantified using the precipitate from which ethanol was completely removed. The precipitate was dissolved in 30 ml of tertiary distilled water, and the colanic acid was quantified using 1 ml of the dissolved precipitate. According to a previous reference (Ren G et al (2016) J Bacteriol. 198(11): 1576-1584; Blumenkrantz N et al (1973) Anal Biochem. 54(2): 484-489), a titer of final colanic acid was determined by mainly counting glucuronic acid among the monosaccharides that constitute colanic acid. 5 ml of 12.5 mM sodium tetraborate dissolved in a 95% sulfuric acid solution was added to 1 ml of the precipitate dissolved in tertiary distilled water, immersed in boiling water at 100 C for 5 minutes, and then placed on ice and sufficiently cooled. 100 μl of a solution of 1.5 g/L hydroxydiphenyl dissolved in 0.5% (w/v) sodium hydroxide was added thereto and the resulting mixture was sufficiently well mixed. A titer of colanic acid was determined using the value measured at 526 nm.

### [Example 4] Cell Culture Conditions in Optimized Medium

For culture experiments, a single colony was selected from a Luria-Bertani (LB) agar plate containing ampicillin, inoculated into 10 mL of an LB culture solution containing ampicillin, and cultured in an incubator at 37° C. and 200 rpm for 12 hours. Then, in order to collect a large amount of cells, the colony was transferred to 100 mL of an LB culture medium containing ampicillin, and the cells were additionally cultured at 37° C. and 200 rpm for 16 hours. The medium used during culturing consisted of 20 g/L glucose, 10.62 g/L sodium phosphate dibasic, 3 g/L potassium phosphate, 0.5 g/L sodium chloride, 2.63 g/L tryptone, 0.24 g/L magnesium sulfate and 0.011 g/L calcium chloride, and is a medium optimized for maximally producing colanic acid according to Korean Patent Application No. 10-2019-0049226.

### [Example 5] Changes in Production Amount of Colanic Acid by Introducing resF Gene

In order to examine the effect of waaF gene deletion and rcsF gene expression on the production of colanic acid, an experiment was performed using bacteria in which the rcsF gene was additionally introduced into E. coli in which the waaF gene was deleted. In terms of the growth curve, recombinant E. coli showed lower growth than the parental strain E. coli JM109 (DE3) and produced a relatively large amount of colanic acid.

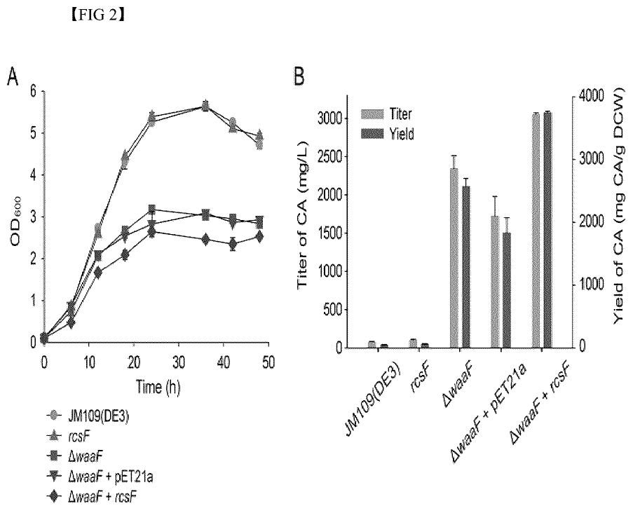

When an rcsF gene was additionally introduced into E. coli in which the waaF gene was deleted, the concentration and yield of colanic acid were about 1.4-fold and about 1.2-fold higher, respectively, than those of colanic acid produced by the E. coli in which the waaF gene was deleted (FIG. 2). Based on this result, the E. coli ΔwaaF+rcsF strain into which the waaF gene deletion and the rcsF gene were additionally introduced was used in an experiment for the production of colanic acid in the present invention.

### [Example 6] Effect of Osmotic Stress Using Sodium Chloride on Production of Colanic Acid

Colanic acid is synthesized in response to changes in the lipopolysaccharide structure of the outer membrane. An experiment was performed using sodium chloride to examine how osmotic stress affects the colanic acid produced by recombinant E. coli. A fermentation experiment was performed by adding sodium chloride at various concentrations in a range of 0 to 300 mM to the optimized medium.

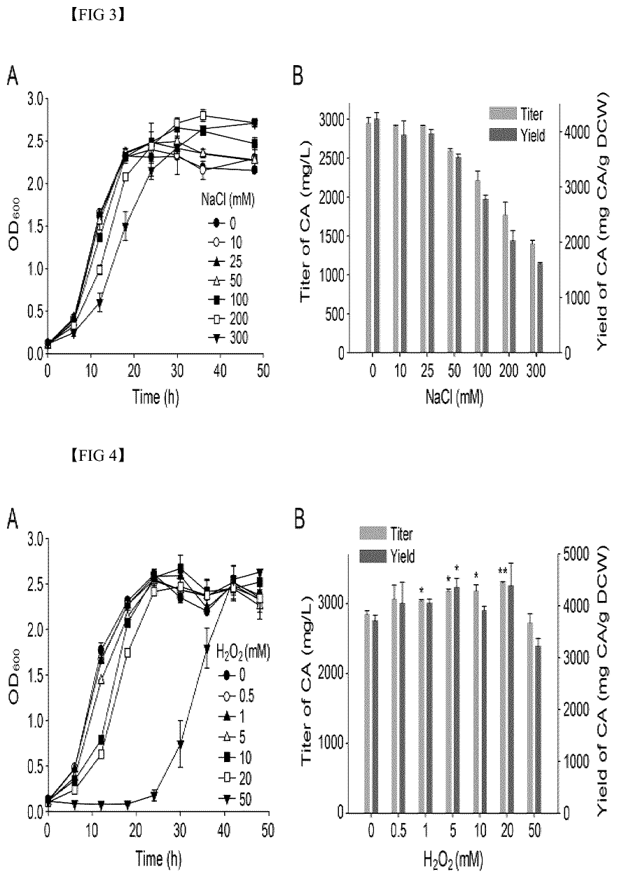

The result shows that as the concentration of sodium chloride increased, rather than producing colanic acid as a defense mechanism against the increase in sodium chloride, the production amount of colanic acid decreased (FIG. 3). As a result of estimating the relevant reason, it was predicted that the deletion of the waaF gene already caused the deformation of the cell membrane, and therefore, when additional osmotic stress was applied, the cell membrane was excessively stressed, thereby leading to apoptosis. In the end, sodium chloride did not help increase the production of colanic acid, but rather gave the effect of decreasing the production of colanic acid.

### [Example 7] Effect of Oxidative Stress Using Hydrogen Peroxide on Production of Colanic Acid

To examine the effect of oxidative stress on the production of colanic acid by the E. coli ΔwaaF+rcsF strain, hydrogen peroxide was added to the medium at each concentration, and then the pattern of the colanic acid produced by fermentation was examined. In the present experiment, hydrogen peroxide at a concentration of 0.5, 1, 5, 10, 20 and 50 mM was added to the medium to observe the growth curve of recombinant E. coli and changes in the concentration and yield of colanic acid. As a result, it was confirmed that the concentration of colanic acid increased 1.2-fold in the presence of 20 mM hydrogen peroxide when compared with a control containing no hydrogen peroxide (FIG. 4). Unlike osmotic stress, oxidative stress was found to increase the production of colanic acid.

### [Example 8] Effect of pH Conditions on Production of Colanic Acid

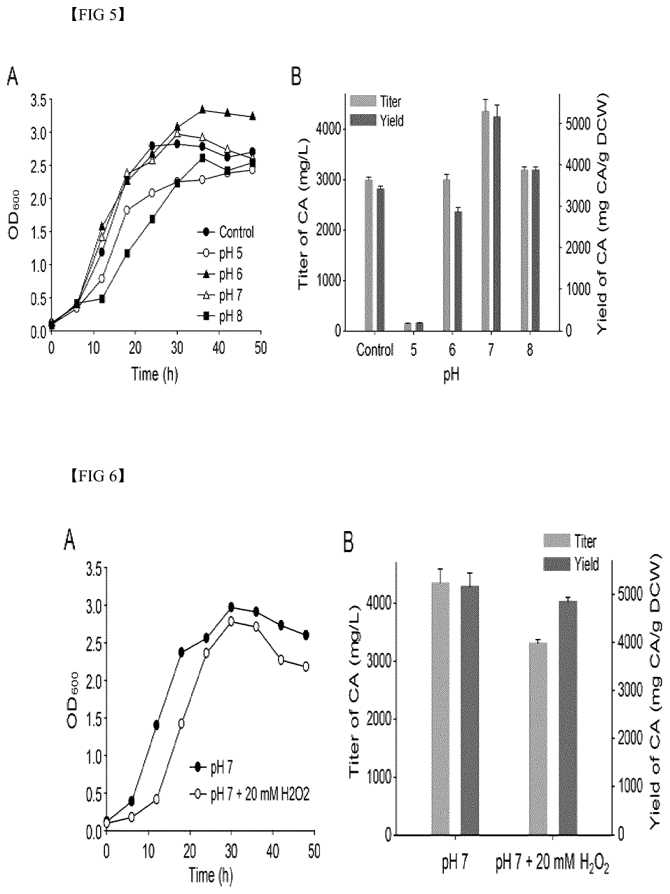

An experiment was performed using a 1-L fermenter to determine an optimum pH for obtaining maximum colanic acid. 5 M hydrochloric acid and 5 M sodium hydroxide were used such that the pH values were maintained at 5, 6, 7 and 8. When compared with other pH conditions through the growth curve, it was found that the growth of the E. coli ΔwaaF+rcsF strain was relatively inhibited at pH 5 and pH 8 (FIG. 5). This is because pH 5 and 8 are out of the optimum pH range for the growth of E. coli. Among various pH conditions, the titer of colanic acid produced by recombinant E. coli grown at pH 7 was the highest at 4351.6 mg/L (FIG. 5). The concentration was about 1.4-fold higher than that of the control bacteria whose pH was not adjusted. In conclusion, maximum colanic acid was produced when the medium was maintained at pH 7. Through this experiment, it was proved that among environmental factors, pH is an important factor that greatly increases the production of colanic acid.

Since the conditions of pH 7.0 and 20 mM hydrogen peroxide maximized the production amount of colanic acid among the above three different environmental factors, it was confirmed whether a synergistic effect could be given to the production of colanic acid when the two conditions were simultaneously applied. As a result, the production amount of colanic acid when the two conditions were simultaneously applied was rather lower than that produced when only the pH 7.0 condition was given alone (FIG. 6).

Through the present invention, the genetic and environmental factors that affect the production of colanic acid and the concentration and yield of colanic acid obtained under each condition are summarized (FIG. 7). E. coli JM109 (DE3) was used as a parental strain to compare the amounts of colanic acid produced by various changes in genetic and environmental factors in the previous invention and the present invention. As an experimental control, the titer and yield of colanic acid were observed to be 79.5 mg/L and 45.2 mg/g, respectively. By removing waaF from E. coli JM109 (DE3), the concentration and yield of colanic acid were observed to be 211.6 mg/L and 460.4 mg/g DCW, respectively. After a culture medium composition was optimized to increase the production of colanic acid of E. coli ΔwaaF, the concentration and yield of colanic acid were 2052.8 mg/L and 2234.3 mg/g DCW, respectively. Thereafter, when the E. coli ΔwaaF+rcsF strain into which rcsF was additionally introduced was cultured in the optimized medium, the concentration and yield of the colanic acid produced were 3051.2 mg/L and 3744.1 mg/g DCW, respectively.

Finally, when the E. coli ΔwaaF+rcsF strain was cultured under pH 7 conditions in the present invention, colanic acid with a maximum concentration and yield of colanic acid of 4351.6 mg/L and 5180.4 mg/g DCW was obtained. In particular, 5180.4 mg/g DCW, which is the yield of colanic acid obtained in the present invention, is the largest value reported to date, and the meaning thereof is significant.

Additionally, as a result of culturing the strain in an incubator (a 250-mL flask or a 1-L fermenter (bioreactor)) by varying culture volumes, it was confirmed that the smaller the culture volume, the higher the production yield of colanic acid. Specifically, it was confirmed that when the strain was fermented with a culture volume of 50 mL in a 250-mL flask and when the strain was fermented with a culture volume of 300 mL in a 1-L fermenter (bioreactor), colanic acid was produced in the highest yield. (FIG. 8)

The method for producing colanic acid of the present invention optimizes environmental factors such as the genetic factors of the E. coli strain and the components of the culture medium, pH, and stress so as to be suitable for production of colanic acid, and the concentration and yield of colanic acid were remarkably increased compared to before optimization.

1. A method for producing colanic acid, the method comprising: preparing a recombinant E. coli JM109 strain by removing a waaF gene from an E. coli JM109 strain and introducing an rcsF gene; and
culturing the prepared recombinant E. coli JM109 strain in a fermentation medium.

2. The method of claim 1, wherein the recombinant E. coli JM109 strain is cultured in a fermentation medium under conditions of pH 7.

3. The method of claim 1, wherein the culturing step comprises producing oxidative stress by adding an oxidizing agent.

4. The method of claim 3, wherein the oxidizing agent is selected from the group consisting of hydrogen peroxide (H₂O₂), a superoxide ion (O₂_{^{−}}), a hydroxyl radical (OH⁻), hypochlorous acid (HOCl) and combinations thereof.

5. The method of claim 1, wherein the culturing step is performed in an incubator, and a culture volume is 10 to 50% of the total volume of the incubator.

6. The method of any one of claim 1, wherein the fermentation medium comprises glucose, tryptone and sodium phosphate (Na₂HPO₄).

7. The method of claim 6, wherein the fermentation medium further comprises sodium chloride (NaCl), magnesium sulfate (MgSO₄), calcium chloride (CaCl₂)) and potassium phosphate (KH₂PO₄).

8. The method of claim 7, wherein the fermentation medium comprises 10 to 30 g/l glucose, 7 to 15 g/l sodium phosphate, 1 to 5 g/l potassium phosphate, 0.1 to 1 g/l sodium chloride, 1 to 5 g/l tryptone, 0.1 to 0.5 g/l magnesium sulfate and 0.005 to 0.02 g/l calcium chloride.


SEQUENCE LISTING (4 sequences)

SEQ ID NO: 1 - DNA, 72 bp, synthetic construct
  misc_feature 1..72: note = waaF forward primer
  source 1..72: mol_type = other DNA
atggtgccgtccattattatcgcggatgccggaagttaacgaagctattcttgtgtaggctggagctgcttc

SEQ ID NO: 2 - DNA, 70 bp, synthetic construct
  misc_feature 1..70: note = waaF reverse primer
  source 1..70: mol_type = other DNA
gataaccctccgcagcgtcacctttacgcactttgtgatagccggtaatcatgggaattagccatggtcc

SEQ ID NO: 3 - DNA, 32 bp, synthetic construct
  misc_feature 1..32: note = rcsF-N primer
  source 1..32: mol_type = other DNA
aaaggatccatgcgtgctttaccgatctgttt

SEQ ID NO: 4 - DNA, 32 bp, synthetic construct
  misc_feature 1..32: note = rcsF-C primer
  source 1..32: mol_type = other DNA
aaactcgagtcatttcgccgtaatgttaagcg